(19) United States
(12) Patent Application Publication
(10) Pub. No.: US 2011/0110824 A1
(43) Pub. Date: May 12, 2011

(54) AROMATIC DEVICE

(76) Inventor: Ming Jen Hsiao, Toufen (TW)

(21) Appl. No.: 12/614,563

(22) Filed: Nov. 9, 2009

(51) Int. Cl. A61L 9/03

(52) U.S. Cl. 422/125

(57) ABSTRACT

An aromatic device has a base, a timing control circuit board, a hollow housing, at least two electric heaters and an aromatic container. The timing control circuit board is mounted inside the base and connected with an external power source. The hollow housing shields the base and has a touch slot for fixing and accessing to operate a power switch. The aromatic container is lap-jointed on the hollow housing and has two separately isolated aromatic chambers respectively receiving an aromatic material therein. Each of the electric heaters heats the corresponding aromatic chamber continuously or on schedule so as to generate continuous or scheduled fragrance or mix up new fragrance. The touch slot is recessed into the hollow housing to prevent accidental touch of the function selection button and enhance safety.

### FIELD OF THE INVENTION

The present invention relates to an aromatic device, and more particularly to an aromatic device generating at least two fragrances.

### BACKGROUND OF THE INVENTION

Conventional aromatherapy diffuser only provides single fragrance and fails to change or mix fragrances. Such limitation easily makes users reluctant to consume more as a result of loss of freshness. Meanwhile, it is inconvenient for the conventional aromatherapy diffuser having single container of aromatic material to change aromatic material for generating different smell of aromatic material. Accordingly, changing aromatic material and smell of the conventional aromatherapy diffuser is ineffective.

Regularly, conventional aromatherapy diffuser does not provide the functions of scheduled heating or continuous heating of aromatic material. Conventional electric heater directly heats aromatic container. As the electric heater is smaller than the aromatic container and is built with different material from that of the aromatic container, the heat conducted over the aromatic container is uneven and it takes longer time to get the aromatic container heated.

Besides, the switches of the aforementioned aromatherapy diffusers for activating or powering on/off the electric heater are all mounted on an outer periphery thereof, making it prone to accidental heating due to careless contact.

### SUMMARY OF THE INVENTION

In view of a same fragrance blunting the smell of users, the present invention provides several fragrances to be switched over and mixed up a new fragrance to transform or excite users' emotion.

In accordance with the present invention, the timing control circuit board can simultaneously control a plurality of heaters to heat the aromatic materials inside a plurality of aromatic containers so as to sequentially or simultaneously radiate different fragrances or blend into a different fragrance at one time to create a strong smell with personal style so as to arouse the insensitive sense of smell just like the cocktail or perfume is mixed up.

A main objective of the present invention is to provide an aromatic device scheduled to heat aromatic material and diffuse fragrance.

Another objective of the present invention is to provide an aromatic device preventing accidental touch on power switch, thereby enhancing the operational safety.

To achieve the foregoing objectives, an aromatic device is provided. The aromatic device has a base, a timing control circuit board, a hollow housing, at least two electric heaters, and an aromatic container.

The base has a control hole and multiple indicator holes. The timing control circuit board is mounted inside the base and adapted to be connected with an external power supply. The hollow housing is lap-jointed on the base to shield the base. Each of the at least two electric heaters has a supporting body, a heater and a heat conductor. The supporting body is mounted inside the hollow housing and fixedly connected with the base. The heater is mounted on the supporting body and electrically connected with the timing control circuit board to generate heat with power supplied by the timing control circuit board. The heat conductor is contacted with the heater. The aromatic container is lap-jointed on the hollow housing, and has at least two aromatic chambers. Each of the aromatic chambers receives an aromatic material and is contacted with the heat conductor of a corresponding electric heater.

The aromatic device further has a function selection button mounted inside the control hole of the base, and touched to link the actuator of the timing control circuit board. When the bottom case is mounted under the hollow housing, the function selection button is mounted inside the touch slot to prevent the actuator from being accidentally touched.

Instead of directly heating the aromatic container, the heater of the corresponding electric heater conducts heat through the heat conductor to more uniformly and rapidly heat the aromatic material inside the aromatic container.

The foregoing and other features and advantages of the present invention will be more clearly understood through the following descriptions with reference to the drawing, wherein:

### BRIEF DESCRIPTION OF THE DRAWINGS

FIG. 1 is a perspective view of a first preferred embodiment of a aromatic device in accordance with the present invention;

FIG. 2 is an exploded perspective view in FIG. 1;

FIG. 3 is another exploded perspective view in FIG. 1;

FIG. 4 is a cross-sectional view in FIG. 1.

### DETAILED DESCRIPTION OF THE PREFERRED EMBODIMENT

The present invention will now be described more specifically with reference to the following embodiments. The following descriptions of preferred embodiments of this invention are presented herein for the purpose of illustration and description only. It is not intended to be exhaustive or to be limited to the precise form disclosed.

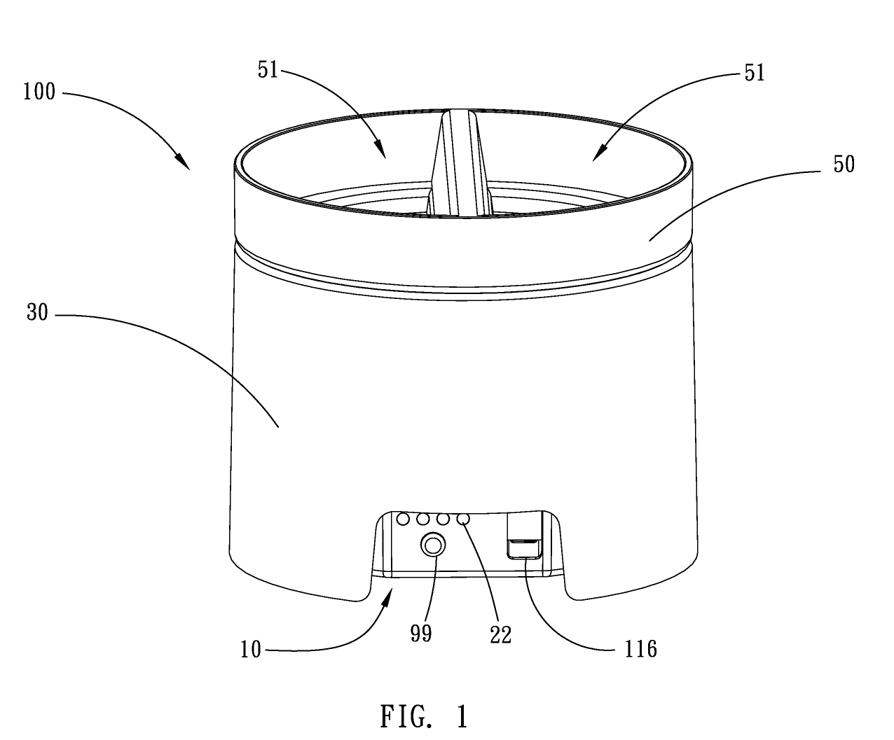

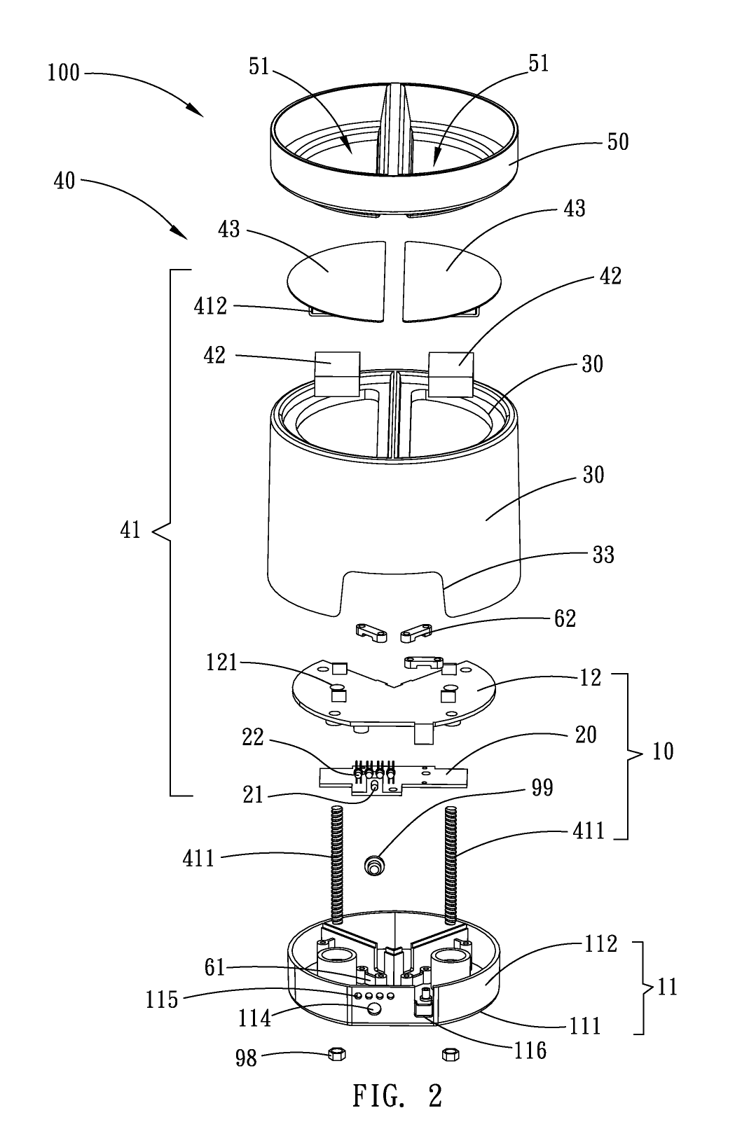

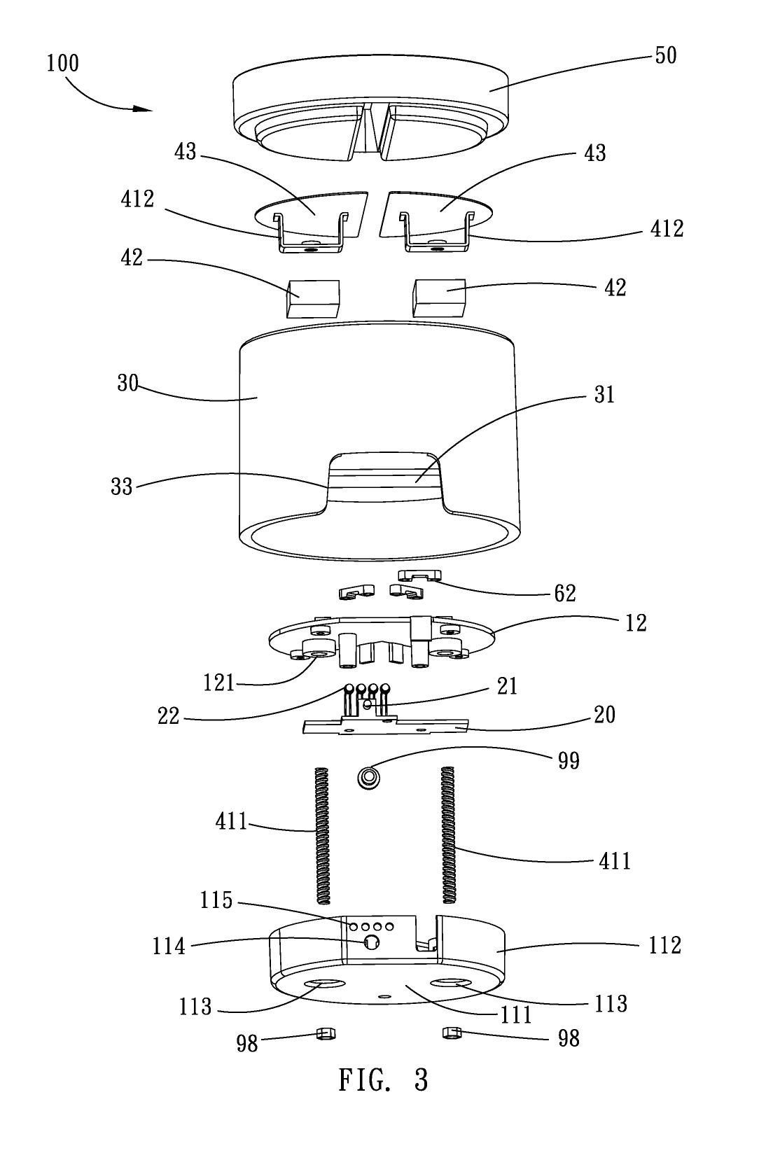

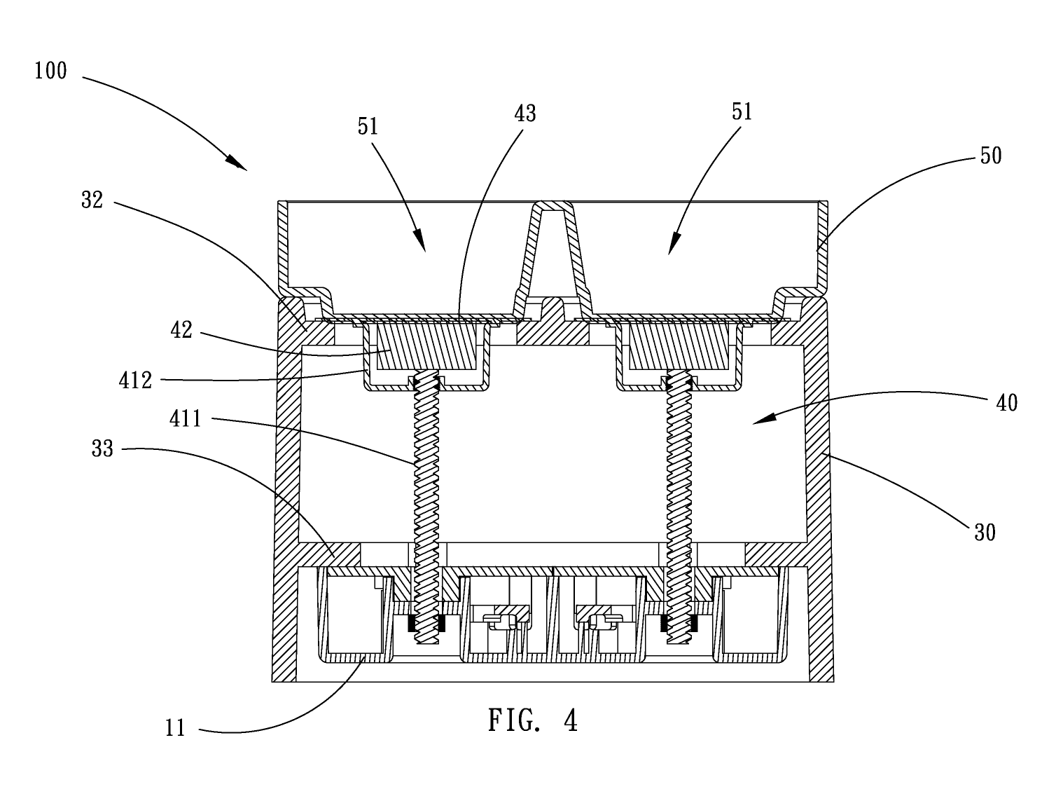

With reference to FIGS. 1 to 4, an aromatic device (100) of an embodiment of the present invention has a base (10), a timing control circuit board (20), a hollow housing (30), two electric heaters (40) and an aromatic container (50).

The base (10) is steadily placed on a surface. The timing control circuit (20) is mounted in the base (10) and is electrically connected with an external power source. The hollow housing (30) is lap jointed on the base (10) to shield the base (10). Each of the electrical heaters (40) has a supporting body (41), a heater (42) and a heat conductor (43). The supporting body (41) is located inside the hollow housing (30) and is fixedly connected with the base (10). The heater (42) is mounted on the corresponding supporting body, is heated by the electrically connected power source, and is electrically connected with the timing control circuit (20). The power source supplies power to the heater (42) through the timing control circuit board (20). The heat conductor (43) is connected with the corresponding supporting body (41) and is contacted with the corresponding heater (42) so that heat generated by the heater (42) is uniformly spread all over the heat conductor (42).

The aromatic container (50) is recessed from a top thereof to form two aromatic chambers (51) which are independently isolated. The aromatic container (50) is mounted inside the hollow housing (30). A bottom of each of the aromatic chambers (51) is in touch with the corresponding heat conductor (43) of a corresponding electric heater (40). Heat generated by each heater (42) is uniformly conducted to the corresponding aromatic chamber (51) through the corresponding heat conductor (43). Thus aromatic materials, such as, fragrant wax and essential oil, placed inside the aromatic chambers (51) can be sequentially or simultaneously heated in accordance with the setting of the timing control circuit board (20) so that different fragrances are generated from the aromatic materials inside the aromatic chambers or a particular fragrance is mixed up therefrom.

Despite two aromatic chambers (51) formed inside the aromatic device of the present invention, the number of the aromatic chamber (51) could be three, four or five (not shown). In that case, the number of the electric heater (40) could be three, four or five (not shown) corresponding to that of the aromatic chamber (51). Different aromatic materials placed in the electric heaters are sequentially, simultaneously or separately heated by supplying power to the respective electric heaters through the timing control circuit board (20) so as to provide multiple fragrances at different times or blend into a fragrance at a time. Therefore, personally favorite fragrance or smell can be generated to dedicate for transforming or exciting user's emotion.

Hence, users can control the timing control circuit board (20) to activate the electric heater (40). The aromatic materials inside the aromatic container (50) are heated to radiate fragrance for users to enjoy the smell. When a scheduling function of the timing control circuit board (20) is used to turn on or off the electric heater (40), such function can also replace noisy ring to wake up users with smell. Instead of a regular audible alarm, the aromatic device is treated as a smell alarm, easing up the shock when users are woken up.

The base (10) further has a control hole (114), multiple indicator holes (115) and a power opening (116). The timing control circuit board (20) has an actuator (21) and multiple LED indicators (22). Each of the LED indicators (22) penetrates through and is exposed beyond the corresponding indicator hole (115) of the base (10). The actuator (21) corresponds to the control hole (114) of the base (10). A power cable (not shown) is inserted in the power opening (116) to connect with the timing control circuit board (20) so as to supply the required power. Therefore, the timing control circuit board (20) is controlled by pressing the actuator (21) to supply power to the electric heaters (40). Each of the LED indicators (22) displays the corresponding heating status. When a first LED indicator is lit, this signals that one of the electric heaters is on. When a second LED indicator is lit, this signals that the other of the electric heaters is on. When a third LED indicator is lit, this signals that a time switch is on. When a fourth LED indicator is lit, this signals that a time switch is off.

The timing control circuit board (20) can sequentially, simultaneously or separately supplies power to the corresponding electric heater(s) (40) to generate heat. When the aromatic material inside an aromatic container (51) is separately heated by a corresponding electric heater (40), a single fragrance is generated. When the timing control circuit board is scheduled to supply power to the respective electric heaters (40), the aromatic materials inside the aromatic chambers (51) are sequentially heated to generate different fragrances. When the aromatic materials inside the aromatic chambers (51) are heated simultaneously, all resulting fragrances are mixed up to generate a unique fragrant smell, thereby increasing peculiarity of fragrance and enhancing user-perceived freshness. When a single electric heater (40) is activated, two electric heaters (40) are simultaneously activated, or any one electric heater (40) is activated and deactivated at scheduled times, the aromatic materials inside the aromatic container (50) are respectively heated by the electric heaters to radiate fragrance.

The base (10) further has a bottom case (11) and a top cover (12). The bottom case (11) has a bottom wall (111) and an annular wall (112) extended upwardly from a rim of the bottom wall (111). A hollow-out chamber is defined within the bottom wall (111) and the annular wall (112). The bottom wall (111) has two holes (113) formed through the bottom wall (111) and communicating with the chamber. The annular wall (112) has the control hole (114), the indicator holes (115) and the power opening (116) formed through the annular wall (112) and communicating with the chamber. The top cover (12) is covered on the bottom case (11), and has two through holes (121) respectively corresponding to the holes (113). Each of the supporting bodies (41) of the corresponding electric heater is fixedly connected on the base (10) by passing through the corresponding through hole (121) and the corresponding hole (113).

The timing control circuit board (20) may be mounted inside the chamber of the base (10).

The present invention further has a function selection button (99) mounted inside the control hole (114) of the base (10). The function selection button (99) is pressed to link the actuator (21) of the timing control circuit board (20). A touch slot (33) is formed through a bottom periphery of the hollow housing (30), and the control hole (114), the indicator holes (115), the power opening (116) and the function selection button (99) corresponds to and conceal the touch slot (33) when combined therein. As the actuator (21) of the timing control circuit board (20) and the function selection button (99) are all hidden inside the touch slot (33) of the hollow housing (30) by adopting a concealed design instead of an exposed design, the power button won't be accidentally touched by children or other external force, thereby increasing operational safety.

The hollow housing (30) has a lower flange (31) annularly protruded beyond a bottom end of an inner wall of the hollow housing (30), and an upper flange (32) annularly protruded beyond a top end of the inner wall. The base (12) is lap-jointed on the lower flange (32), and the aromatic container (50) is lap jointed on the upper flange (32).

One end of the supporting body (41) is a bolt (411) passing through the corresponding through holes (121) and is fastened on the corresponding hole (113) by a nut (98). The other end of the supporting body (41) is a support tray for placing the corresponding electric heater (42) thereon.

The present invention further has multiple wire holders (61) and fasteners (62). Each of the wire holders (61) is mounted on the bottom wall (111) of the base (10) for receiving electric wires. Each of the fasteners (62) is snapped on the corresponding wire holder (61) to fix and receive the corresponding electric wire so as not to mess up the electric wires.

Instead of being directly heated by the heater of the corresponding electric heater (40), the aromatic container (50) is heated through the conduction of the heat conductor (43) so that the aromatic material inside the corresponding aromatic container (50) is more uniformly and rapidly heated up.

The heater (42) of the corresponding electric heater (40) may be a positive temperature coefficient thermistor (PTC) or a cement wire wound resistor.

The heat conductor (43) of the corresponding electric heater (40) is made of a metal with better thermal conductivity.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims, which are to be accorded with the broadest interpretation so as to encompass all such modifications and similar structures.

What is claimed is:

1. An aromatic device, comprising:
a base;
a timing control circuit board mounted inside the base and adapted to be connected with an external power supply;
a hollow housing lap jointed on the base to shield the base;
at least two electric heaters, each of the electric heaters having
a supporting body mounted inside the hollow housing and fixedly connected with the base;
a heater mounted on the supporting body and electrically connected with the timing control circuit board to generate heat with power supplied by the timing control circuit board; and
a heat conductor contacted with the heater; and
an aromatic container lap-jointed on the hollow housing, and having at least two aromatic chambers, each of the aromatic chambers receiving an aromatic material and contacted with the heat conductor of a corresponding electric heater.

2. The aromatic device as claimed in claim 1, wherein the timing control circuit board sequentially, simultaneously or separately supplies power to the heater of each of the electric heaters.

3. The aromatic device as claimed in claim 1, wherein the base further comprises a control hole, multiple indicator holes and a power opening, the timing control circuit board has an actuator and multiple LED indicators, the indicators respectively pass through the indicator holes, the actuator corresponds to the control hole to be actuated to control the timing control circuit, and the power opening is adapted for an external electric wire to be inserted therein and electrically connected the control circuit board.

4. The aromatic device as claimed in claim 1, further comprising a function selection button mounted inside the control hole of the base, and touched to link the actuator of the timing control circuit board.

5. The aromatic device as claimed in claim 4, wherein a touch slot is formed through a bottom periphery of the hollow housing to correspond to and conceal the touch slot when combined therein.

6. The aromatic device as claimed in claim 1, wherein the base has a bottom case and a top cover, the bottom case has a bottom wall and an annular wall extended upwardly from a rim of the bottom wall, a hollow-out chamber is defined within the bottom wall and the annular wall for receiving the timing control circuit board, and the top cover is covered on the bottom case.

7. The aromatic device as claimed in claim 6, wherein the bottom wall has two holes formed through the bottom wall and communicating with the chamber; the annular wall has the control hole, the indicator holes and the power opening formed through the annular wall and communicating with the chamber, the top cover has two through holes respectively corresponding to the holes, each of the supporting bodies of the corresponding electric heater is fixedly connected on the base by passing through the corresponding through hole and the corresponding hole.

8. The aromatic device as claimed in claim 1, wherein the hollow housing has a lower flange annularly protruded beyond a bottom end of an inner wall of the hollow housing, and an upper flange annularly protruded beyond a top end of the inner wall, the base is lap jointed on the lower flange, and the aromatic container is lap-jointed on the upper flange.

9. The aromatic device as claimed in claim 1, wherein one end of the supporting body is a bolt fastened on the base, and the other end of the supporting body is a support tray for placing the corresponding electric heater thereon to contact with the heat conductor.

10. The aromatic device as claimed in claim 1, wherein the heater of the corresponding electric heater is a positive temperature coefficient thermistor or a cement wire wound resistor.
